(19) United States
(12) Patent Application Publication
(10) Pub. No.: US 2021/0174560 A1
(43) Pub. Date: Jun. 10, 2021

(54) SCATTER CORRECTION FOR LONG AXIAL FOV

(71) Applicant: Siemens Medical Solutions USA, Inc., Malern, PA (US)

(72) Inventors: Inki Hong, Knoxville, TN (US); Ziad Burbar, Knoxville, TN (US); Stefan B. Siegel, Knoxville, TN (US)

(21) Appl. No.: 16/702,824

(22) Filed: Dec. 4, 2019

(51) Int. Cl. G06T 11/00; A61B 6/03; A61B 6/04; A61B 6/00; G01T 1/161; G06T 7/00; G01T 1/17

(52) U.S. Cl. CPC G06T 11/005; A61B 6/037; A61B 6/032; A61B 6/0407; A61B 6/5205; G06T 2207/30048; G06T 7/0012; G01T 1/17; G06T 2207/10104; G06T 2207/10081; G06T 2207/30016; G01T 1/161

(57) ABSTRACT

A computer-implemented method for scatter correction includes receiving a nuclear imaging data set, generating a scatter-estimation from the nuclear imaging data set using a ring-specific singles countrate, and generating a clinical image incorporating the scatter-estimation.

### TECHNICAL FIELD

This application relates generally to nuclear imaging and, more particularly, to long axial field-of-view nuclear imaging.

### BACKGROUND

Current short axial field-of-view (FOV) scanner axial extents vary by less than some amount, such as, for example, about 21 cm (+/−5 cm) and have imaging diameters of about 80 cm. For scanner systems having a short axial FOV (referred to as short axial FOV scanner systems), the same distribution of a singles rate at a detector for a given organ and radiotracer may be assumed without regard to geometrical differences. When using radiotracer compounds with a short half-life, e.g., O-15, Rb-82, etc., a patient may be injected with a very high dose so that the short axial FOV is able to collect a sufficient quantity of data (e.g., statistics) to generate reconstructions. For scanner systems having a long axial FOV (referred to as long axial FOV scanner systems), the spread in singles countrates is significant over the length of the long axial FOV.

Current systems use a singles countrate to characterize a shift in the signal amplitude per detected event. PET scanners are run with an energy window applied to a detected photon. As the single countrate increases, there is a corresponding shift in signal amplitude per event, which is detected as a shift in the lower level discrimination (LLD) of the energy window. The effective shift in LLD as a function of a system's mean singles countrate is characterized and used as a parameter for scatter estimate. Current solutions cannot be used for systems having significant spread in singles countrates.

### SUMMARY

In various embodiments, a computer-implemented method for scatter correction is disclosed. The computer-implemented method includes steps of receiving a nuclear imaging data set, generating a scatter-estimation from the nuclear imaging data set using a ring-specific singles countrate, and generating a clinical image incorporating the scatter-estimation.

In various embodiments, a system is disclosed. The system includes a nuclear imaging scanner and a computer. The computer is configured to receive a nuclear imaging data set from the nuclear imaging scanner and generate a scatter-estimation from the nuclear imaging data.

In various embodiments, a non-transitory computer readable medium storing instructions is disclosed. The instructions are configured to cause a computer system to execute the steps of receiving a nuclear imaging data set, generating a scatter-estimation from the nuclear imaging data set using a ring-specific singles countrate, and generating a clinical image incorporating the scatter-estimation.

### BRIEF DESCRIPTION OF THE FIGURES

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a nuclear imaging system, in accordance with some embodiments.

FIG. 2 illustrates a block diagram of a computer system, in accordance with some embodiments.

FIG. 3 illustrates organ activity distribution for a brain and a heart with respect to a long-axial FOV system and a short axial FOV system, in accordance with some embodiments.

FIG. 4 is a chart illustrating a singles per block distribution for a long axial FOV system, in accordance with some embodiments.

FIG. 5 is a flowchart illustrating a method of scatter correction incorporating block singles countrate, in accordance with some embodiments.

FIG. 6 illustrates the trajectories of scatter gamma rays, in accordance with some embodiments.

FIG. 7A is a chart illustrating the average singles per block for a short axial FOV system.

FIG. 7B is a chart illustrating the average singles per block for a long axial FOV system, in accordance with some embodiments.

### DETAILED DESCRIPTION

The description of the exemplary embodiments is intended to be read in connection with the accompanying drawings, which are to be considered part of the entire written description. In the description, relative terms such as “lower,” “upper,” “horizontal,” “vertical,” “proximal,” “distal,” “above,” “below,” “up,” “down,” “top” and “bottom,” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

As used herein, the term “substantially” denotes elements having a recited relationship (e.g., parallel, perpendicular, aligned, etc.) within acceptable manufacturing tolerances. For example, as used herein, the term “substantially parallel” is used to denote elements that are parallel or that vary from a parallel arrangement within an acceptable margin of error, such as +/−5°, although it will be recognized that greater and/or lesser deviations can exist based on manufacturing processes and/or other manufacturing requirements.

In various embodiments, systems and methods of performing scatter correction including ring-specific singles countrates are disclosed. Nuclear imaging data is obtained by an imaging modality. The nuclear imaging data is provided to a system configured to perform scatter correction and generate a clinical image, such as a 3D sinogram. The nuclear imaging data is scatter corrected using a ring-specific singles countrate, such as, for example, a block ring average, an individual block average, an individual crystal average, etc. After performing scatter correction, a diagnostic image, such as a 3D sinogram, is generated from the scatter corrected nuclear imaging data.

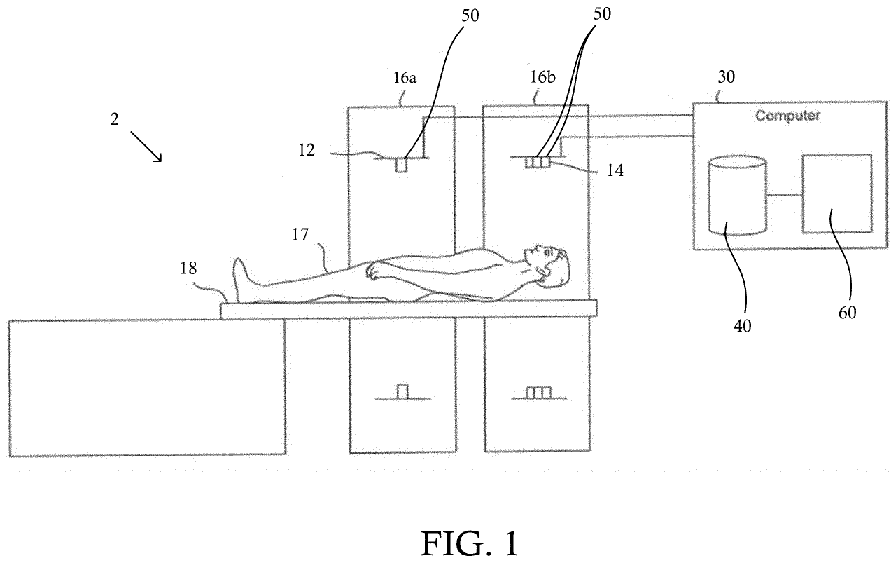

FIG. 1 illustrates one embodiment of a nuclear imaging system 2. The nuclear imaging system 2 includes at least a first imaging modality 12 provided in a first gantry 16a. The first imaging modality 12 may include any suitable modality, such as, for example, a computed-tomography (CT) modality, a positron-emission tomography (PET) modality, a single-photon emission computerized tomography (SPECT) modality, etc. The first imaging modality 12 may include a long axial FOV or a short axial FOV. A patient 17 lies on a movable patient bed 18 that may be movable with respect to the first gantry 16a. In some embodiments, the nuclear imaging system 2 includes a second imaging modality 14 provided in a second gantry 16b. The second imaging modality 14 can be any suitable imaging modality, such as, for example, a CT modality, a PET modality, a SPECT modality and/or any other suitable imaging modality. The second modality 14 may include a long axial FOV or a short axial FOV. Each of the first imaging modality 12 and/or the second imaging modality 14 can include one or more detectors 50 arranged, for example, in one or more rings. Each of the detectors 50 is configured to detect an annihilation photon, gamma ray, and/or other nuclear imaging event.

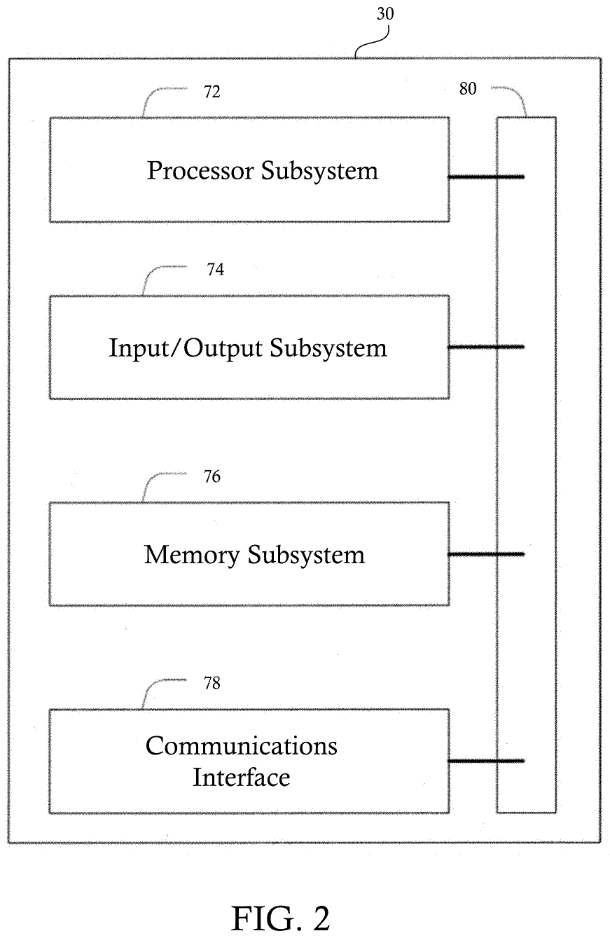

Scan data from the first imaging modality 12 and/or the second imaging modality 14 is stored at one or more computer databases 40 and processed by one or more computer processors 60 of a computer system 30. The graphical depiction of computer system 30 in FIG. 1 is provided by way of illustration only, and computer system 30 may include one or more separate computing devices, for example, as described with respect to FIG. 2. The scan data may be provided by the first imaging modality 12, the second imaging modality 14, and/or may be provided as a separate data set, such as, for example, from a memory coupled to the computer system 30. The computer system 30 can include one or more processing electronics for processing a signal received from one of the plurality of detectors 50.

FIG. 2 illustrates a computer system 30 configured to implement one or more processes, in accordance with some embodiments. The system 30 is a representative device and can include a processor subsystem 72, an input/output subsystem 74, a memory subsystem 76, a communications interface 78, and a system bus 80. In some embodiments, one or more than one of the system 30 components can be combined or omitted such as, for example, not including an input/output subsystem 74. In some embodiments, the system 30 can comprise other components not shown in FIG. 2. For example, the system 30 can also include, for example, a power subsystem. In other embodiments, the system 30 can include several instances of a component shown in FIG. 2. For example, the system 30 can include multiple memory subsystems 76. For the sake of conciseness and clarity, and not limitation, one of each component is shown in FIG. 2.

The processor subsystem 72 can include any processing circuitry operative to control the operations and performance of the system 30. In various aspects, the processor subsystem 72 can be implemented as a general purpose processor, a chip multiprocessor (CMP), a dedicated processor, an embedded processor, a digital signal processor (DSP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The processor subsystem 72 also can be implemented by a controller, a microcontroller, an application specific integrated circuit (ASIC), a field programmable gate array (FPGA), a programmable logic device (PLD), and so forth.

In various aspects, the processor subsystem 72 can be arranged to run an operating system (OS) and various applications. Examples of an OS comprise, for example, operating systems generally known under the trade name of Apple OS, Microsoft Windows OS, Android OS, Linux OS, and any other proprietary or open source OS. Examples of applications comprise, for example, network applications, local applications, data input/output applications, user interaction applications, etc.

In some embodiments, the system 30 can include a system bus 80 that couples various system components including the processing subsystem 72, the input/output subsystem 74, and the memory subsystem 76. The system bus 80 can be any of several types of bus structure(s) including a memory bus or memory controller, a peripheral bus or external bus, and/or a local bus using any variety of available bus architectures including, but not limited to, 9-bit bus, Industrial Standard Architecture (ISA), Micro-Channel Architecture (MSA), Extended ISA (EISA), Intelligent Drive Electronics (IDE), VESA Local Bus (VLB), Peripheral Component Interconnect Card International Association Bus (PCMCIA), Small Computers Interface (SCSI) or other proprietary bus, or any custom bus suitable for computing device applications.

In some embodiments, the input/output subsystem 74 can include any suitable mechanism or component to enable a user to provide input to system 30 and the system 30 to provide output to the user. For example, the input/output subsystem 74 can include any suitable input mechanism, including but not limited to, a button, keypad, keyboard, click wheel, touch screen, motion sensor, microphone, camera, etc.

In some embodiments, the input/output subsystem 74 can include a visual peripheral output device for providing a display visible to the user. For example, the visual peripheral output device can include a screen such as, for example, a Liquid Crystal Display (LCD) screen. As another example, the visual peripheral output device can include a movable display or projecting system for providing a display of content on a surface remote from the system 30. In some embodiments, the visual peripheral output device can include a coder/decoder, also known as Codecs, to convert digital media data into analog signals. For example, the visual peripheral output device can include video Codecs, audio Codecs, or any other suitable type of Codec.

The visual peripheral output device can include display drivers, circuitry for driving display drivers, or both. The visual peripheral output device can be operative to display content under the direction of the processor subsystem 72. For example, the visual peripheral output device can be able to play media playback information, application screens for application implemented on the system 30, information regarding ongoing communications operations, information regarding incoming communications requests, or device operation screens, to name only a few.

In some embodiments, the communications interface 78 can include any suitable hardware, software, or combination of hardware and software that is capable of coupling the system 30 to one or more networks and/or additional devices. The communications interface 78 can be arranged to operate with any suitable technique for controlling information signals using a desired set of communications protocols, services or operating procedures. The communications interface 78 can include the appropriate physical connectors to connect with a corresponding communications medium, whether wired or wireless.

Vehicles of communication comprise a network. In various aspects, the network can include local area networks (LAN) as well as wide area networks (WAN) including without limitation Internet, wired channels, wireless channels, communication devices including telephones, computers, wire, radio, optical or other electromagnetic channels, and combinations thereof, including other devices and/or components capable of/associated with communicating data. For example, the communication environments comprise in-body communications, various devices, and various modes of communications such as wireless communications, wired communications, and combinations of the same.

Wireless communication modes comprise any mode of communication between points (e.g., nodes) that utilize, at least in part, wireless technology including various protocols and combinations of protocols associated with wireless transmission, data, and devices. The points comprise, for example, wireless devices such as wireless headsets, audio and multimedia devices and equipment, such as audio players and multimedia players, telephones, including mobile telephones and cordless telephones, and computers and computer-related devices and components, such as printers, network-connected machinery, and/or any other suitable device or third-party device.

Wired communication modes comprise any mode of communication between points that utilize wired technology including various protocols and combinations of protocols associated with wired transmission, data, and devices. The points comprise, for example, devices such as audio and multimedia devices and equipment, such as audio players and multimedia players, telephones, including mobile telephones and cordless telephones, and computers and computer-related devices and components, such as printers, network-connected machinery, and/or any other suitable device or third-party device. In various implementations, the wired communication modules can communicate in accordance with a number of wired protocols. Examples of wired protocols can include Universal Serial Bus (USB) communication, RS-232, RS-422, RS-423, RS-485 serial protocols, FireWire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, to name only a few examples.

Accordingly, in various aspects, the communications interface 78 can include one or more interfaces such as, for example, a wireless communications interface, a wired communications interface, a network interface, a transmit interface, a receive interface, a media interface, a system interface, a component interface, a switching interface, a chip interface, a controller, and so forth. When implemented by a wireless device or within wireless system, for example, the communications interface 78 can include a wireless interface comprising one or more antennas, transmitters, receivers, transceivers, amplifiers, filters, control logic, and so forth.

In various aspects, the communications interface 78 can provide data communications functionality in accordance with a number of protocols. Examples of protocols can include various wireless local area network (WLAN) protocols, including the Institute of Electrical and Electronics Engineers (IEEE) 802.xx series of protocols, such as IEEE 802.11a/b/g/n/ac, IEEE 802.16, IEEE 802.20, and so forth. Other examples of wireless protocols can include various wireless wide area network (WWAN) protocols, such as GSM cellular radiotelephone system protocols with GPRS, CDMA cellular radiotelephone communication systems with 1×RTT, EDGE systems, EV-DO systems, EV-DV systems, HSDPA systems, and so forth. Further examples of wireless protocols can include wireless personal area network (PAN) protocols, such as an Infrared protocol, a protocol from the Bluetooth Special Interest Group (SIG) series of protocols (e.g., Bluetooth Specification versions 5.0, 6, 7, legacy Bluetooth protocols, etc.) as well as one or more Bluetooth Profiles, and so forth. Yet another example of wireless protocols can include near-field communication techniques and protocols, such as electro-magnetic induction (EMI) techniques. An example of EMI techniques can include passive or active radio-frequency identification (RFID) protocols and devices. Other suitable protocols can include Ultra Wide Band (UWB), Digital Office (DO), Digital Home, Trusted Platform Module (TPM), ZigBee, and so forth.

In some embodiments, at least one non-transitory computer-readable storage medium is provided having computer-executable instructions embodied thereon, wherein, when executed by at least one processor, the computer-executable instructions cause the at least one processor to perform embodiments of the methods described herein. This computer-readable storage medium can be embodied in memory subsystem 76.

In some embodiments, the memory subsystem 76 can include any machine-readable or computer-readable media capable of storing data, including both volatile/non-volatile memory and removable/non-removable memory. The memory subsystem 8 can include at least one non-volatile memory unit. The non-volatile memory unit is capable of storing one or more software programs. The software programs can contain, for example, applications, user data, device data, and/or configuration data, or combinations therefore, to name only a few. The software programs can contain instructions executable by the various components of the system 30.

In various aspects, the memory subsystem 76 can include any machine-readable or computer-readable media capable of storing data, including both volatile/non-volatile memory and removable/non-removable memory. For example, memory can include read-only memory (ROM), random-access memory (RAM), dynamic RAM (DRAM), Double-Data-Rate DRAM (DDR-RAM), synchronous DRAM (SDRAM), static RAM (SRAM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, disk memory (e.g., floppy disk, hard drive, optical disk, magnetic disk), or card (e.g., magnetic card, optical card), or any other type of media suitable for storing information.

In one embodiment, the memory subsystem 76 can contain an instruction set, in the form of a file for executing various methods, such as methods including A/B testing and cache optimization, as described herein. The instruction set can be stored in any acceptable form of machine readable instructions, including source code or various appropriate programming languages. Some examples of programming languages that can be used to store the instruction set comprise, but are not limited to: Java, C, C++, C #, Python, Objective-C, Visual Basic, or .NET programming In some embodiments a compiler or interpreter is comprised to convert the instruction set into machine executable code for execution by the processing subsystem 72.

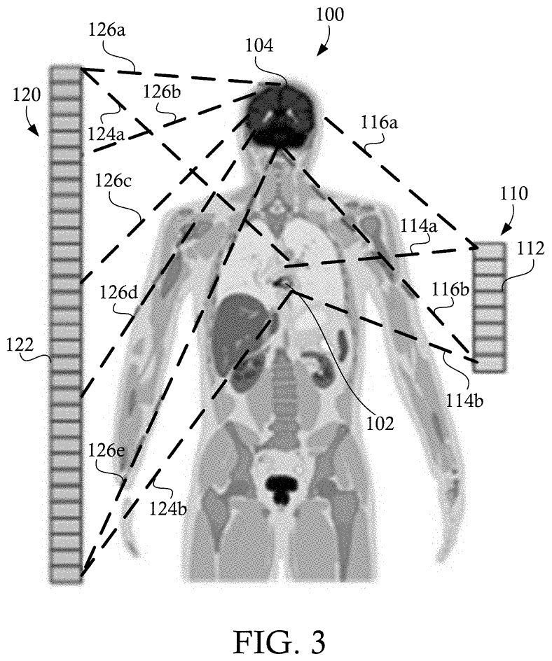

FIG. 3 illustrates organ activity distribution for a first organ 102 and a second organ 104 with respect to a short axial FOV scanner system 110 and a long-axial FOV scanner system 120, in accordance with some embodiments. During scanning, a patient 100 is positioned on a bed, such as, for example, bed 18 illustrated in FIG. 1. In a short axial FOV scanner system 110, the detector 112 has a short FOV that detects a first set events 114a, 114b originating from within a first organ 102, such as the heart and a second set of events 116a, 116b originating from within a second organ 104, such as a brain. The short FOV limits the number of events detected from either the first organ 102 or the second organ 104, requiring a large dose of radiotracer (e.g., radioactive isotope) to generate adequate data for reconstruction generation. In contrast, the long axial FOV scanner system 120 includes a detector 122 extending over a larger portion of a patient 100, for example, from the head to the mid-thighs. The detector 122 has a long axial FOV that detects a first set of events 124a, 124b occurring in the first organ 102 and a second set of events 126a-126e occurring in the second organ 104. The number of events (i.e., the singles countrate) in the long axial FOV is greater than the number of events in the short axial FOV. It should be noted that for illustration purposes, in FIG. 3, the detectors 112 and 122 are shown in a cross-sectional view sectioned through the patient's coronal plane and only one side of the detector rings that make up the detectors 112 and 122 are illustrated.

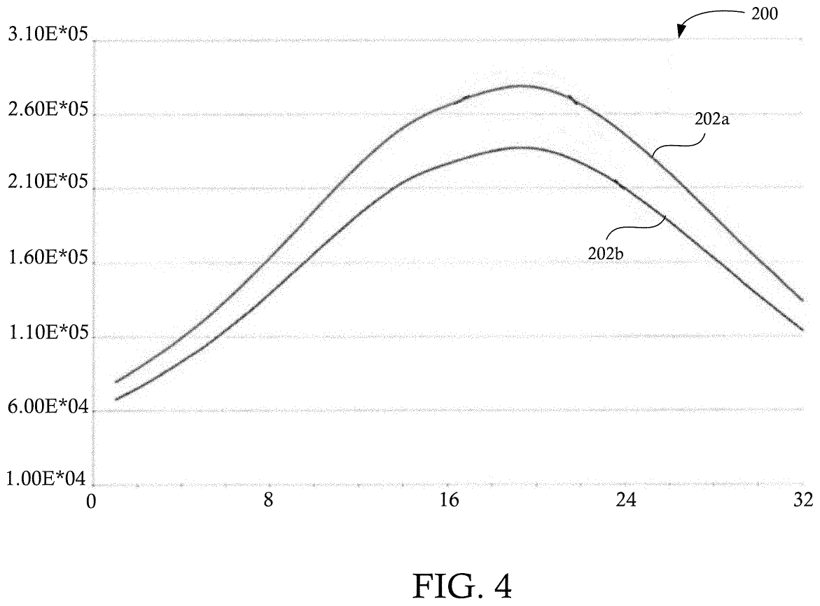

FIG. 4 is a chart 200 illustrating singles per block distributions 202a, 202b for long axial FOV systems, in accordance with some embodiments. Each of the singles per block distributions 202a, 202b have a low distribution for block numbers at the edges of the long axial FOV (e.g., detectors located near the ends of the detector 122 illustrated in FIG. 3). The singles per block count increases towards the middle of the detector 122, for example, corresponding to the portion of the detector 122 positioned adjacent to a target organ. The singles per block distributions 202a, 202b illustrate that a global average of singles per block cannot be applied to the long axial FOV scanner 120.

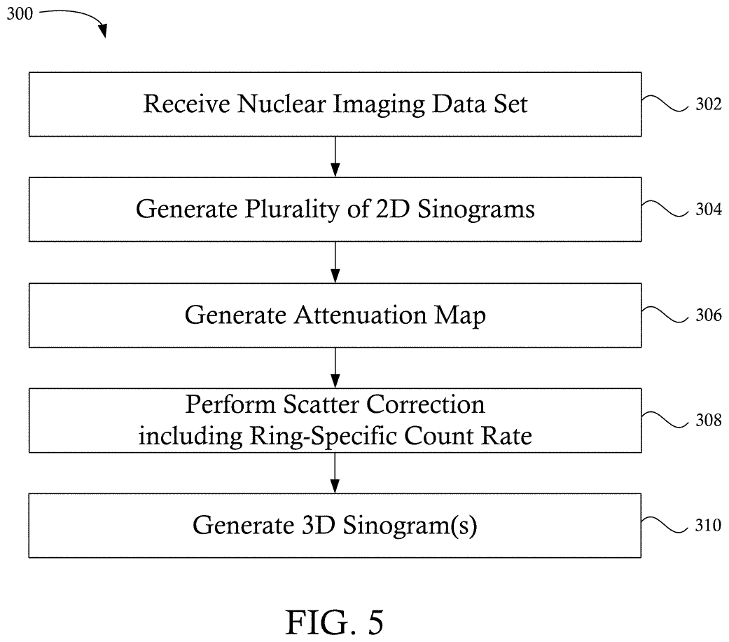

FIG. 5 is a flowchart 300 illustrating a method of scatter correction using a ring-specific singles countrate, in accordance with some embodiments. At step 302, a nuclear imaging data set is received at a system, such as, for example, computer 30. The nuclear imaging data set may be received from any suitable nuclear imaging data source, such as, for example, directly from an imaging modality 12 (or 14), a memory unit, and/or any other suitable source. The nuclear imaging data source can be such as, for example, a PET imaging modality, a CT imaging modality, a SPECT imaging modality, and/or any other suitable imaging modality.

At step 304, a plurality of 2D sinograms are generated from the nuclear imaging data. The 2D sinograms may be generated using any suitable method. For example, in some embodiments, 2D sinograms are generated from estimates of the emitter and attenuation distributions without scatter correction from direct plane data. Although specific embodiments are discussed herein, it will be appreciated that the 2D sinograms may be generated using any suitable method. At step 306, an attenuation map is generated for the nuclear imaging data based on the imaging modality used to collect the nuclear imaging data. The attenuation map may be generated using any suitable method known in the art. The attenuation map is configured to provide scatter probabilities with respect to the long axial FOV of the detector 122.

At step 308, scatter correction is performed. Scatter correction may include, but is not limited to, a Monte Carlo and/or other analytic process. The process may be configured to receive the ring-specific singles countrate, the attenuation map, the plurality of 2D sinograms, and/or other suitable data for performing an estimation of potential outcomes within the analytical process (e.g., potential scatter corrected sinograms). In some embodiments, the scatter correction generates a scatter matrix or other output useable during reconstruction of a clinical image.

In some embodiments, the ring-specific singles countrate (e.g., block ring average, an individual block average, an individual crystal average. etc.) is configured to account for variation in singles rates due to scanner geometry (which is not accounted for when using a global singles rate). As the countrate increases, detector signals may shift (e.g., amplitude may shift). The amplitude shift may manifest as an effective shift in the lower level discrimination (LLD) of the energy window of the detector. The shift in LLD causes an effect on the number of counts for a specific ring and effects the angle of a detected event. The ring-specific singles countrate corrects for the shifts in LLD.

The ring-specific singles countrate may be provided at any suitable granularity for which ring countrate information is available. For example, in various embodiments, the singles countrate may include, but is not limited to, block ring average, individual block average, individual crystal average, and/or any other suitable granularity. It will be appreciated that a ring-specific singles countrate having a higher granularity (e.g., individual block average, individual crystal average, etc.) will be more time and computation intensive than a ring-specific singles countrate having a lower granularity (e.g., block ring average, individual block average, etc.). The selection of granularity of the ring-specific singles countrate may be based on available computing power, available computing time, required resolution of a final image, and/or any other suitable factors.

In some embodiments, the 2D sinograms are arranged into projection views at varying azimuthal angles, direct axial angle segments, and oblique axial angle segments For each sampled projection view, a uniform two-dimensional group of line-of-response (LOR) samples may be defined. For each combination of sample LOR and scatter sample point in the object, a scatter contribution to the LOR may be computed. The ring-specific singles countrate are incorporated into the scatter contribution calculation to account for a shift in LLD.

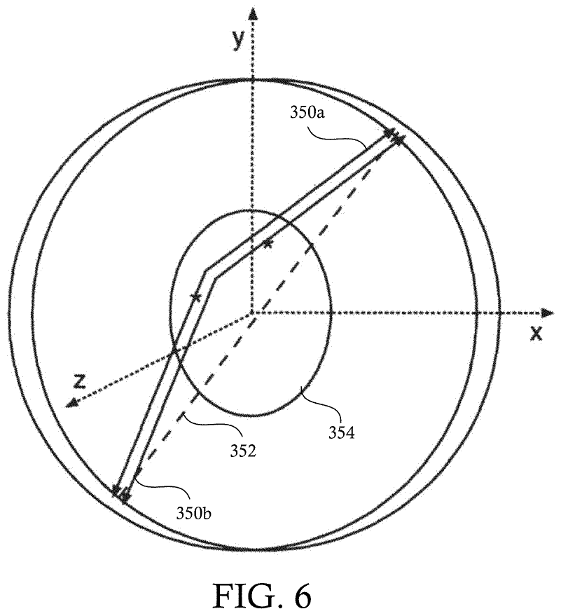

FIG. 6 illustrates the trajectories 350a-350b of scatter gamma rays, in accordance with some embodiments. A LOR 352 is contained in the projection view at some azimuthal and polar angle. For each scatter point in the scan volume 354, there will be two distinct contributions to the singly scattered coincidences in the LOR 352, depending where on the scatter point the emission point lies. The total coincidence rate in the LOR 352 due to singly scattered events can be expressed as a volume integral. In some embodiments, one or more efficiency factors may be adjusted and/or determined based on a ring-specific singles countrate to account for the angle incidence, penetration, and energy resolution and discrimination of a detector 50. Although specific embodiments are discussed herein, it will be appreciated that the use of ring-specific singles countrate may be applied to any suitable scatter correction process to account for a shift in LLD for each detector ring, each individual detector, each individual crystal, etc. depending on the selected resolution of the ring-specific singles countrate.

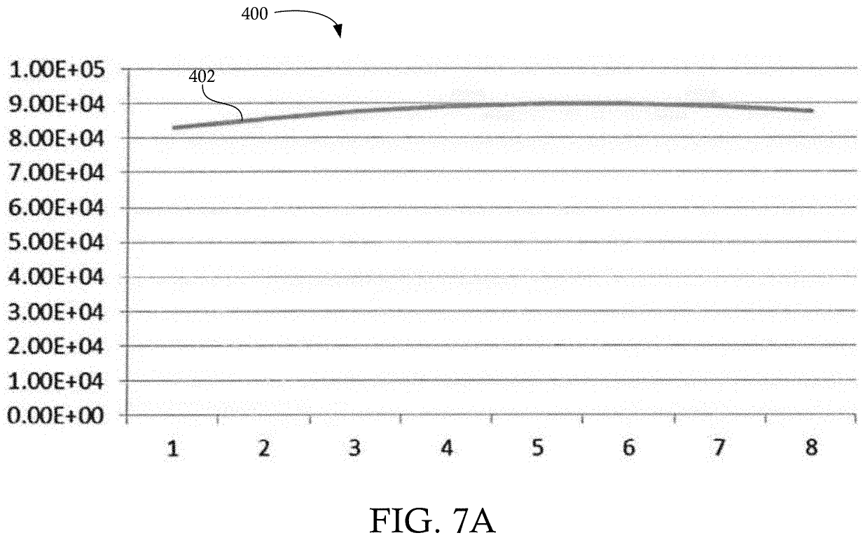

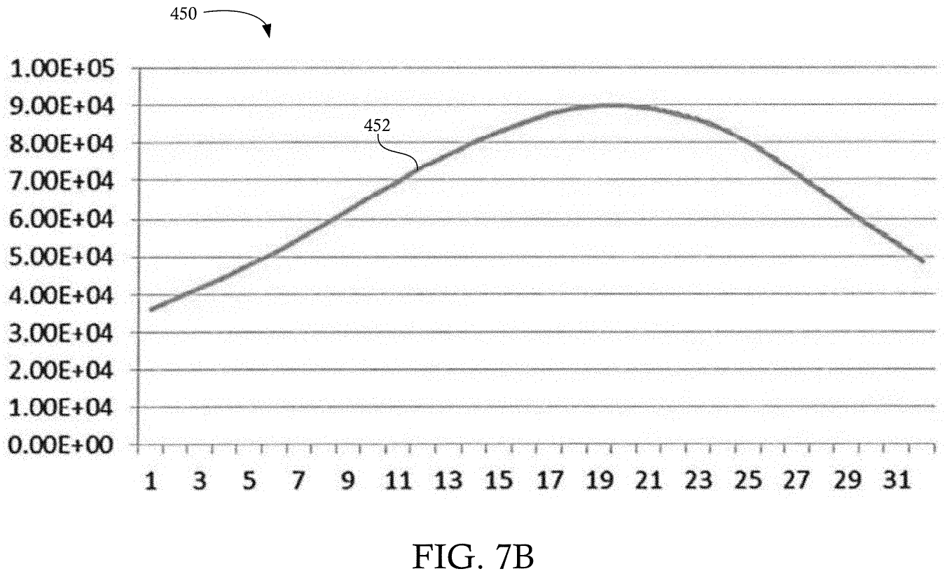

FIG. 7A is a chart 400 illustrating the average singles per block 402 for a short axial FOV scanner system and FIG. 7B is a chart 450 illustrating the average singles per block 452 for a long axial FOV scanner system, in accordance with some embodiments. As illustrated in FIGS. 7A and 7B, the singles count per detector ring varies from detector ring to detector ring within a scanner. The long axial FOV scanner system, as illustrated in chart 450, has a greater variance due to the presence of a greater number of detector rings over a larger axial distance. The LLD of each ring within both the short axial FOV scanner system and the long axial FOV scanner system is compensated by the ring-specific singles countrate value provided to the scatter correction process. It will be appreciated that although the variance of the average singles countrate for the short axial FOV scanner system is much lower than the variance for the long axial FOV scanner system, scatter correction in both the short axial FOV scanner system and the long axial FOV scanner system is improved using the ring-specific singles countrate.

With reference back to FIG. 5, at step 310, a clinical image, such as a 3D sinogram, is generated based on the scatter-corrected 2D sinograms. The 3D sinogram may be output for review by a clinician, stored in memory, transmitted to a remote system, and/or otherwise stored for review. The 3D sinogram may be generated from the plurality of scatter-corrected 2D sinograms using any suitable process. For example, in various embodiments, the 3D sinogram is generated using a modality-specific process based on the imaging modality used to generate the nuclear image data.

Although the subject matter has been described in terms of exemplary embodiments, it is not limited thereto. Rather, the appended claims should be construed broadly, to include other variants and embodiments, which may be made by those skilled in the art.

What is claimed is:

1. A computer-implemented method, comprising:
receiving a nuclear imaging data set;
generating scatter-estimation of the nuclear imaging data set using a ring-specific singles countrate; and
generating a clinical image incorporating the scatter-estimation.

2. The computer-implemented method of claim 1, wherein the ring-specific singles countrate comprises an average singles per block for each ring in a detector.

3. The computer-implemented method of claim 1, wherein the ring-specific singles countrate comprises an individual block average for each block in a ring.

4. The computer-implement method of claim 1, further comprising generating a plurality of 2D sinograms prior to generating the scatter-estimation.

5. The computer-implemented method of claim 1, wherein the scatter-estimation is generated using a Monte Carlo process or an analytical process.

6. The computer-implemented method of claim 1, wherein the nuclear imaging data set is generated by a long-axial field of view scanner.

7. The computer implemented method of claim 1, wherein the ring-specific singles countrate is configured to estimate a shift in a lower level discriminator of an energy window of the nuclear imaging data set.

8. A system, comprising:
a nuclear imaging scanner; and
a computer configured to:
receive a nuclear imaging data set from the nuclear imaging scanner;
generate a scatter estimation of the nuclear imaging data set using a ring-specific singles countrate.

9. The system of claim 8, wherein the ring-specific singles countrate comprises an average singles per block for each detector ring in the nuclear imaging scanner.

10. The system of claim 8, wherein the ring-specific singles countrate comprises an individual block average for each block in each detector ring in the nuclear imaging scanner.

11. The system of claim 8, wherein the computer is configured to generate a plurality of 2D sinograms prior to generating the scatter estimation.

12. The system of claim 8, wherein the scatter estimation is generated using a Monte Carlo process or an analytical process.

13. The system of claim 8, wherein the nuclear imaging scanner is a long-axial field of view scanner.

14. The system of claim 8, wherein the ring-specific singles countrate is configured to estimate a shift in a lower level discriminator of an energy window of the nuclear imaging scanner.

15. A non-transitory computer readable medium storing instructions configured to cause a computer system to execute the steps of:
receiving a nuclear imaging data set;
generating a scatter-estimation of the nuclear imaging data set using a ring-specific singles countrate; and
generating a clinical image incorporating the scatter-estimation.

16. The non-transitory computer readable medium of claim 15, wherein the ring-specific singles countrate comprises an average singles per block for each ring in a detector.

17. The non-transitory computer readable medium of claim 15, wherein the ring-specific singles countrate comprises an individual block average for each block in a ring.

18. The non-transitory computer readable medium of claim 15, wherein the instructions cause the computer system to execute the step of generating a plurality of 2D sinograms prior to generating the scatter estimation.

19. The non-transitory computer readable medium of claim 15, wherein the scatter estimation is generated using a Monte Carlo process or an analytical process.

20. The non-transitory computer readable medium of claim 15, wherein the nuclear imaging data set is generated by a long-axial field of view scanner.
